(19) United States
(12) Patent Application Publication
(10) Pub. No.: US 2025/0369045 A1
(43) Pub. Date: Dec. 4, 2025

(54) MULTIVALENT ASSEMBLIES FOR ENHANCED TARGET HYBRIDIZATION

(71) Applicant: Illumina, Inc., San Diebo, CA (US)

(72) Inventors: Carlo Randise-Hinchliff, San Diego, CA (US); Rebekah Karadeema, San Diego, CA (US); Jeffrey Brodin, San Diego, CA (US); Lena Storms, San Diego, CA (US); Jeffrey Fisher, San Diego, CA (US); Andrew Slatter, Cambridge (GB); Sarah Shultzaberger, San Diego, CA (US)

(21) Appl. No.: 18/874,399

(22) PCT Filed: Dec. 19, 2023
(86) PCT No.: PCT/US2023/084945

§ 371 Date: Dec. 12, 2024

Related U.S. Application Data
(60) Provisional application No. 63/476,320, filed on Dec. 20, 2022.

(51) Int. Cl. C12Q 1/6874; C12N 15/10; C12Q 1/6832; C12Q 1/6876

(52) U.S. Cl. CPC C12Q 1/6874; C12N 15/1013; C12N 15/1096; C12Q 1/6832; C12Q 1/6876

(57) ABSTRACT

Multivalent assemblies for target hybridization are described. The multivalent assemblies include oligonucleotide sets that hybridize to a target nucleic acid to permit capture of the target nucleic acid. In an embodiment, the multivalent assemblies are heteromultivalent such that the oligonucleotide sets include different oligonucleotides that bind to different regions of the target nucleic acid.

### CROSS-REFERENCE TO RELATED APPLICATIONS

This application is a national stage application claiming priority to PCT/US23/84945, which claims priority to and the benefit of U.S. Provisional Application No. 63/476,320, entitled “MULTIVALENT ASSEMBLIES FOR ENHANCED TARGET HYBRIDIZATION” and filed on Dec. 20, 2022, the disclosures of which are hereby incorporated by reference in their entireties.

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

Sequencing methodology of next-generation sequencing (NGS) platforms typically makes use of nucleic acid fragment libraries. In targeted sequencing techniques, a subset of fragments containing genes or regions of interest of the genome are isolated from the nucleic acid library and sequenced. Targeted approaches using NGS allow researchers to focus time, expenses, and data analysis on specific areas of interest. Such targeted analysis can include the exome (the protein-coding portion of the genome), specific genes of interest (custom content), targets within genes, or mitochondrial DNA. Targeted approaches contrast with whole genome sequencing approaches that are more comprehensive, but that also involve sequencing regions of the genome that may not be of interest to all users.

In one example of a targeted sequencing technique, target enrichment or hybridization pullout methods use a panel or set of probes that hybridize to target sequences in the nucleic acid library. Hybridization of the probes to the target sequences allows these sequences to be separated from the rest of the fragments in the library to enrich the targeted sequencing using the captured sequences.

### BRIEF DESCRIPTION

In one embodiment, the present disclosure provides a multivalent assembly. The multivalent assembly includes a first single-stranded oligonucleotide probe complementary to a first region of a target nucleic acid, a second single-stranded oligonucleotide probe complementary to a second region of the target nucleic acid, and a third single-stranded oligonucleotide probe complementary to a third region of the target nucleic acid. The first single-stranded oligonucleotide probe, the second single-stranded oligonucleotide probe, and melting temperatures of the first single-stranded oligonucleotide probe, the second single-stranded oligonucleotide probe, and the third single-stranded oligonucleotide probe from the target nucleic acid are all within a 20 degrees Celsius range.

In one embodiment, the present disclosure provides multivalent bead assembly. The multivalent bead assembly includes a bead surface. The multivalent bead assembly also includes first single-stranded oligonucleotide probes comprising a first hybridization sequence complementary to a first region of a target nucleic acid, second single-stranded oligonucleotide probes comprising a second hybridization sequence complementary to a second region of the target nucleic acid, and third single-stranded oligonucleotide probes comprising a third hybridization sequence complementary to a third region of the target nucleic acid. The first single-stranded oligonucleotide probes, the second single-stranded oligonucleotide probes, and the third single-stranded oligonucleotide probes are immobilized randomly on the bead surface, and the first hybridization sequence, the second hybridization sequence and the third hybridization sequence are different from one another.

In one embodiment, the present disclosure provides a hybridization kit. The hybridization kit includes a first bead comprising a first plurality of single-stranded oligonucleotide probes randomly immobilized on a surface of the first bead, wherein a first subset of the first plurality are complementary to a first region of a first target nucleic acid, a second subset of the first plurality are complementary to a second region of the first target nucleic acid, and a third subset are complementary to a third region of the first target nucleic acid. The hybridization kit includes a second bead comprising a second plurality of single-stranded oligonucleotide probes randomly immobilized on a surface of the second bead, wherein a first subset of the second plurality are complementary to a first region of a second target nucleic acid, a second subset of the second plurality are complementary to a second region of the second target nucleic acid, and a third subset of the second plurality are complementary to a third region of the second target nucleic acid.

In one embodiment, the present disclosure provides a method of target enrichment. The method includes fragmenting nucleic acids of a sample to generate nucleic acid fragments comprising target nucleic acids and contacting the nucleic acid fragments with a plurality of multivalent assemblies to form multivalent assembly-target nucleic acid complexes, wherein the multivalent assemblies comprise individual probe sets specific for respective target nucleic acids. An individual probes set includes a first oligonucleotide probe complementary to a first subregion of a target nucleic acid; a second oligonucleotide probe complementary to a second subregion of the target nucleic acid; and a third oligonucleotide probe complementary to a third subregion of the target nucleic acid, wherein the first oligonucleotide probe, the second oligonucleotide probe, and the third oligonucleotide probe have sequences that are distinguishable from one another. The method also includes separating the multivalent assembly-target nucleic acid complexes from unhybridized nucleic acid fragments of the nucleic acid fragments to generate separated nucleic acid fragments.

In one embodiment, the present disclosure provides a method of cDNA synthesis. The method includes contacting an RNA sample with a multivalent assembly to capture an RNA molecule, the multivalent assembly comprising a probe set immobilized on a surface of a bead. The probe set includes a first oligonucleotide probe complementary to a first subregion of the RNA molecule; a second oligonucleotide probe complementary to a second subregion of the RNA molecule; and a third oligonucleotide probe complementary to a third subregion of the RNA molecule. The wherein the first oligonucleotide probe, the second oligonucleotide probe, and the third oligonucleotide probe have sequences that are distinguishable from one another. The method also includes extending the first oligonucleotide probe using a reverse transcriptase to generate a cDNA complementary to at least a portion of the RNA molecule.

The preceding description is presented to enable the making and use of the technology disclosed. Various modifications to the disclosed implementations will be apparent, and the general principles defined herein may be applied to other implementations and applications without departing from the spirit and scope of the technology disclosed. Thus, the technology disclosed is not intended to be limited to the implementations shown, but is to be accorded the widest scope consistent with the principles and features disclosed herein. The scope of the technology disclosed is defined by the appended claims.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 shows a relationship between oligonucleotide length and full-length synthesis;

FIG. 2 shows synthesis failure for an 80-mer oligonucleotide having a hairpin structure;

FIG. 3 is a schematic illustration of a multivalent assembly, in accordance with aspects of the present disclosure;

FIG. 4 shows oligonucleotide probes having different lengths and falling within a melting temperature range;

FIG. 5 is a schematic illustration of an example multivalent assembly having oligonucleotides immobilized on a surface of a bead, in accordance with aspects of the present disclosure;

FIG. 6 is a schematic illustration of an example multivalent assembly having oligonucleotides immobilized on a bead, in accordance with aspects of the present disclosure;

FIG. 7 is a schematic illustration of an example surface of a multivalent assembly having randomly seeded oligonucleotides, in accordance with aspects of the present disclosure;

FIG. 8 is a schematic illustration of an example multivalent assembly including a nucleic acid tile scaffold, in accordance with aspects of the present disclosure;

FIG. 9 is a schematic illustration of an example multivalent assembly including a polypeptide scaffold, in accordance with aspects of the present disclosure;

FIG. 10 shows an example target enrichment workflow using multivalent assemblies, in accordance with aspects of the present disclosure;

FIG. 11 shows an example cell-free nucleic acid capture using multivalent assemblies, in accordance with aspects of the present disclosure;

FIG. 12 shows an example nucleic acid capture using multivalent assemblies with random Nmers, in accordance with aspects of the present disclosure;

FIG. 13 shows an example nucleic acid capture using multivalent assemblies with targeted oligonucleotides, in accordance with aspects of the present disclosure;

FIG. 14 shows an example cDNA extension using multivalent assemblies, in accordance with aspects of the present disclosure;

FIG. 15 shows that the performance of hybridization-based extraction surpasses total nucleic acid extraction;

FIG. 16A shows an experimental workflow to measure the use of melting temperature as a proxy for increased avidity;

FIG. 16B shows images of results of binding decreasing as a function of temperature for fluorescently-tagged targets as in FIG. 16A;

FIG. 17A shows example 1, 3, and 5-probe beads;

FIG. 17B shows melting temperature for 1, 3, and 5-probe beads showing binding coordination for multivalent assemblies with different probe types;

FIG. 18 shows an enrichment protocol suitable for bead enrichment;

FIG. 19A shows example heteromultivalent and monovalent assemblies;

FIG. 19B shows a comparison of target enrichment between heteromultivalent and monovalent assemblies;

FIG. 19C shows a comparison of target enrichment between heteromultivalent and monovalent assemblies;

FIG. 20A shows multivalent bead read enrichment for forward and reverse strands; and

FIG. 20B shows a lambda phage library workflow for multivalent beads versus in-solution probes.

### DETAILED DESCRIPTION

The following discussion is presented to enable any person skilled in the art to make and use the technology disclosed, and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown, but is to be accorded the widest scope consistent with the principles and features disclosed herein.

The use of nucleic acids with specificity for a target sequence may permit target enrichment, amplification, purification, extension, or other reactions. Hybridization probe capture of a subset of nucleic acid sequences from biological samples or from libraries modified with sequencing adapters is used for enrichment in NGS and array-based profiling and for applications such as pathogen detection and disease monitoring. Assays are designed with oligonucleotides probes or binders that have high binding affinity and high specificity to desired analyte relative to other oligonucleotide sequences. Enrichment technologies may employ a workflow that includes hybridization of biotinylated probes to nucleic acid sequences of interest, pulldown of biotinylated probes onto streptavidin-functionalized magnetic beads, washing of beads to remove non-specifically bound molecules, elution of target nucleic acids from beads, and sequencing.

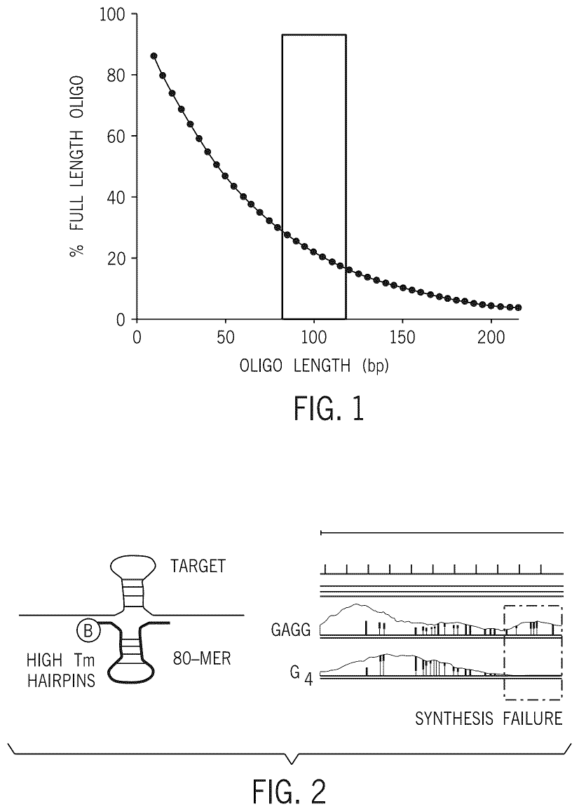

Relatively longer nucleic acids in these assays can provide desired target specificity relative to a shorter nucleic acid and with reduced incidence of off-target binding. The binding strength or avidity of a nucleic acid for a target increases with an increasing number of complementary nucleotides. However, longer nucleic acids used for specific binding reactions are relatively more expensive and complex to synthesize. FIG. 1 shows a relationship between oligonucleotide length and a percentage of manufactured products representing a full-length oligonucleotide. As oligonucleotide length increases, the manufacturing yield of the desired full-length product decreases. In certain cases, contiguous enrichment probes can be ˜80-120 nucleotides. For high throughput oligonucleotide synthesis, stepwise yields may be in the 88-90% range, resulting in final product yields of 15-30% for oligonucleotides of 80-120 bases in length with minimal secondary structure. The synthesis yields are worse for oligonucleotides with higher Tₘ secondary structures like G-quadruplexes, as shown in FIG. 2.

In manufacturing, only full length probes are selected for use in assays. Thus, most of the synthesized product is lost, which makes manufacturing oligonucleotides having 80-120 nucleotides less efficient relative to shorter oligonucleotides. Low probe manufacturing efficiency can result in additional challenges in applications with workflows having a high level of excess oligonucleotide probes relative to sample input. Hybridization capture enrichment applications may use a high library input of 200-500 ng (which may then involve a pre-amplification step) and a high oligonucleotide probe concentration of 2000× molar excess to achieve desired target enrichment. Even with excess probe concentrations, there is variable enrichment due to high temperature secondary structures in these longer probes that lead to poor synthesis and poor capture of target region. Thus, generating sufficient probe concentrations to fulfill the high levels of excess probe in certain workflows can be challenging, particularly for probe lengths associated with low manufacturing yields.

Provided herein are multivalent assemblies, nucleic acids, reagents, kits, probe panels, and methods of manufacturing and using multivalent assemblies. The multivalent assemblies may be part of an isolated reaction or may be used as part of a larger workflow, such as a sequencing workflow. The disclosed techniques provide coordinated shorter oligonucleotides that avoid low target affinity and that have improved synthesis yields as compared to relatively longer probes. Multivalent assemblies are provided that use multiple shorter probes that target a same general region of the target nucleic acid as compared to a single long probe. The present techniques provide cooperatively binding oligonucleotide sets (e.g., probe sets) that include these split or shorter oligonucleotides that bind to different regions of a target nucleic acid. In an embodiment, a target region of the target nucleic acid is made up of shorter subregions targeted by these separate oligonucleotides. This coordination increases the hybridization strength (avidity) with the potential to decrease probe and input requirements, increase the stringency of washes for non-specific dissociation, and avoid potential secondary structures associated with relatively longer probes that lead to lower manufacturing efficiency as well as poor target capture.

In embodiments, the oligonucleotides may be used in conjunction with multivalent surfaces, heteromultivalent surfaces (e.g., beads), or branched oligonucleotides, each of which addresses the problem of inefficient, probe-based capture of nucleic acids. The multivalent assemblies include a set of unique oligonucleotides sequences that are each able to bind to a distinct stretch of nucleic acid region. Multivalent probe structures (such as beads, branched oligonucleotides, or oligonucleotide handles) coordinate binding of separate unique oligonucleotides sequences each able to bind to a distinct stretch of a target. This coordinated hybridization increases the avidity of the multivalent structure to its target without needing to increase the length of each individual probe. The enhanced avidity can also increase the kinetics of hybridization to allow faster annealing times relative to longer probes and reduce the required concentration of the probes and input in the hybridization reaction. In embodiments, using variable length probes within the multivalent assemblies avoids secondary structures and improves uniformity in hybridization strength.

In embodiments, the use of cooperatively binding oligonucleotides of the set provides, in aggregate, binding specificity for the target nucleic acid comparable to a single, contiguous probe spanning a same region of the target nucleic acid. Thus, the manufacturing complexities associated with longer probes may be avoided while avidity is maintained. Further, the present techniques may achieve more efficient probe capture using assemblies that hybridize to both the strands from a nucleic acid duplex but using physically forward and reverse complimentary probes, e.g., separated onto separate beads. Targeting in-solution probes to both strands is more challenging because having both forward and reverse complimentary probes in a reaction solution leads to probe-probe hybridization and a reduced hybridization capture of target region. To avoid probe-probe hybridization of forward and reverse strand probes, certain techniques target only one of the two strands, which makes hybridization capture challenging for lower input and PCR-free libraries. The disclosed techniques can avoid undesired probe-probe hybridization while facilitating targeting of both duplex strands. The disclosed techniques avoid low probe synthesis efficiency for longer length or problematic sequences by decoupling probe sequences from immobilized probes in certain arrangements. That is, by splitting probes into smaller segments, synthesis complexity is avoided but similar hybridization efficiency can be achieved using cooperative binding. In embodiments, the disclosed techniques provide an enhancement in on-target enrichment compared to a contiguous oligonucleotide hybridization capture approach, as shown using the lambda genome as a model system.

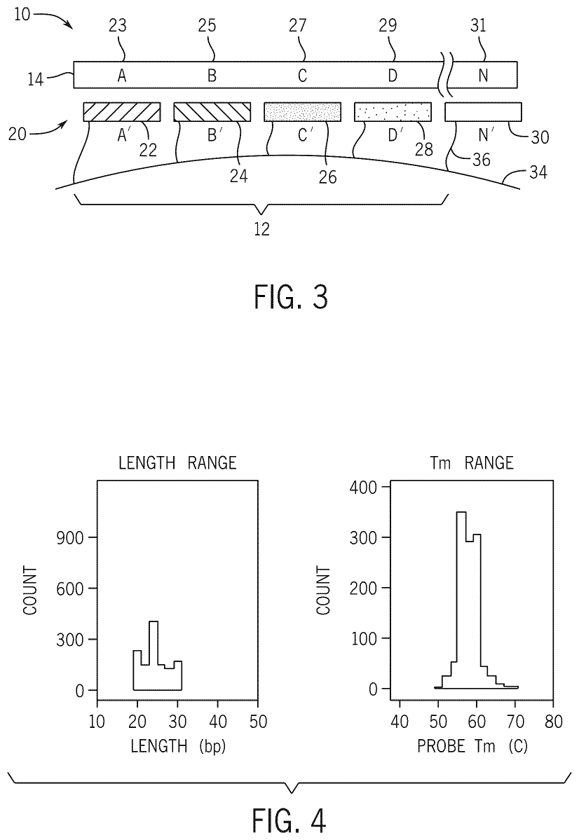

FIG. 3 is a schematic illustration of a multivalent assembly 10 in which a set 12 of oligonucleotides 20 (e.g., oligonucleotide probes) having distinguishable sequences and with binding specificity for a target nucleic acid 14 are immobilized onto a surface 34. The oligonucleotide set 12 includes individual single-stranded oligonucleotides 20 (e.g., single-stranded oligonucleotide probes) that each have complementarity to different regions of the target nucleic acid 14. In an embodiment, the set 12 is an oligonucleotide probe set. As provided herein, the oligonucleotide 20 may refer to an oligonucleotide probe or probe for target enrichment workflows.

As provide herein, the multivalent assembly 10 provides a plurality of hybridization sites or binding sites for a target, such as the target nucleic acid 14. Thus, in an embodiment, multivalent refers to a structure with more than one hybridization or binding site. The individual hybridization sites may include individual oligonucleotides 20 that are separate from one another. Thus, in the illustrated embodiment, individual hybridization sites, shown as A′, B′, C′, D′, and N′, are noncontiguous and are not located on a same oligonucleotide strand. That is, in an embodiment, each individual oligonucleotide 20 that includes a hybridization site complementary to a region of a target nucleic acid 14 has a respective 5′ end and a 3′ end. Thus, as provided herein, separate oligonucleotides 22, 24, 26, 28, and 30 include different individual hybridization sites A′, B′ C′ D′, and N′. As illustrated, the assembly 10 is heteromultivalent, such that oligonucleotides 20 have distinguishable or unique nucleotide sequences relative to other oligonucleotides 20 within the set 12. However, other multivalent arrangements are also contemplated. For example, one or more of the individual oligonucleotides 20 of the set 12 may include a same sequence relative to others of the set 12.

An individual set 12 includes a plurality of separate oligonucleotides 20 that are, respectively, complementary to different regions of the target sequence 14. In the illustrated example, oligonucleotides 22, 24, 26, and 28 (with hybridization sites A′, B′, C′, D′, and N′) represent different oligonucleotides 20 of the set 12. An oligonucleotide 30 represents one or more additional oligonucleotides. The oligonucleotides 22, 24, 26, 28, and 30 are complementary to respective target binding regions 23, 25, 27, 29, and 31 (also denoted as regions A, B, C, D, and N) of the target nucleic acid 14. The target binding regions represent different subregions of the target nucleic acid 14. In an embodiment, the target nucleic acid 14 may be a single-stranded nucleic acid or nucleic acid fragment, and the target binding regions, in total, encompass only a portion of the fragment. That is, the target nucleic acid 14 may include nonhybridizing regions that are not complementary to the oligonucleotides 20. However, binding to the oligonucleotides 20 permits capture and enrichment of the bound fragment, including any nonhybridizing regions.

Each oligonucleotide 20 may be between 10-80 nucleotides in length in an embodiment. By way of example, an individual oligonucleotide 20 of the set 12 may be 10-20 nucleotides in length, 10-30 nucleotides in length, 20-30 nucleotides in length, 10-50 nucleotides in length, or 30-50 nucleotides in length. The complementary target binding regions on the target nucleic acid 14 are relatively shorter (e.g., 10-80 nucleotides in an embodiment) than the binding region of a conventional contiguous or single nucleic acid hybridization probe, which may be 100-300 nucleotides in length. However, in aggregate, the span or length across all of the target binding regions 23, 25, 27, 29, and 31 may be at least 80 nucleotides, 80-150 nucleotides, 80-200 nucleotides, 100-300 nucleotides, or 120-300 nucleotides by way of example. In an embodiment, a full length of the oligonucleotides 20 extending from a 5′ end to a 3′ end is complementary to a respective target binding region. In other embodiments, one or more of the oligonucleotides 20 of the set 12 may include a nonhybridizing region at a 5′ end and/or a 3′ end.

In an embodiment, the oligonucleotides 20 are complementary to directly adjacent or contiguous regions. For example, the target binding regions 23, 25, 27, 29, and 31 may form a continuous span or stretch of the target nucleic acid 14. In other embodiments, a spacer region may be present between one or more of the target binding regions 23, 25, 27, 29, and 31. By way of example, the spacer region may be between 1-10 nucleotides (e.g., 1-4 nucleotides, 1-5 nucleotides) in length. In other examples, the spacer region may be longer to accommodate different multivalent assembly arrangements. For example, a polypeptide base or DNA tile surface 34 may result in oligonucleotides that bind target binding regions that are 10-25 or more nucleotides apart between at least two of the target binding regions. In an embodiment, the target binding regions 23, 25, 27, 29, and 31 are nonoverlapping and arranged in 5′ to 3′ order or vice versa.

In the illustrated example, the oligonucleotide set 12 includes at least four separate oligonucleotides 20. However, it should be understood that more or fewer may be included in the set 12 as generally discussed herein. The number of unique oligonucleotides 20 of the set 12 may depend on variables including the size and shape of the surface 34 and the length of an optional spacer region. For example, in an embodiment, the oligonucleotide set 12 includes a first oligonucleotide, a second oligonucleotide, and a third oligonucleotide.

It should be understood that, in embodiments, the multivalent assemblies 10 may be provided with oligonucleotides 20 in a single-stranded state, e.g., in a nonhybridized or unbound state. However, if a target nucleic acid 14 having a complementary sequence to some or all of the oligonucleotides 20 of the set 12 are present under favorable hybridization conditions (e.g., temperature), the complementary oligonucleotides 20 may hybridize to the target nucleic acid 14. Thus, the oligonucleotides 20, when hybridized to the target nucleic acid 14, are in a duplex or double-stranded state.

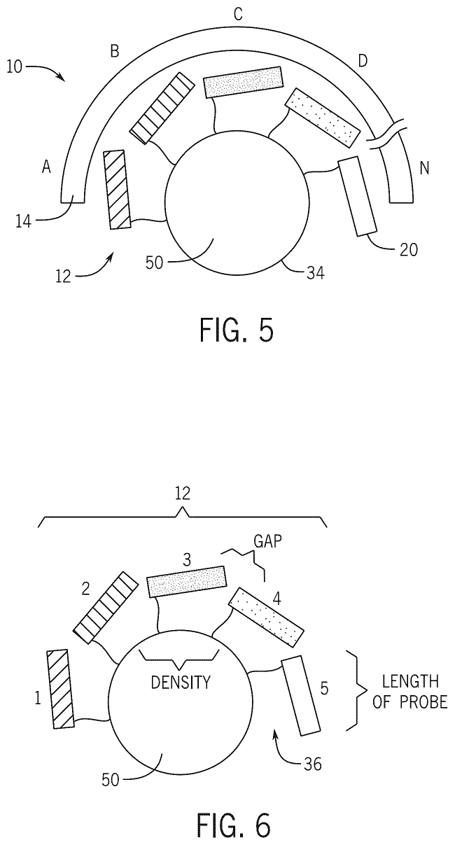

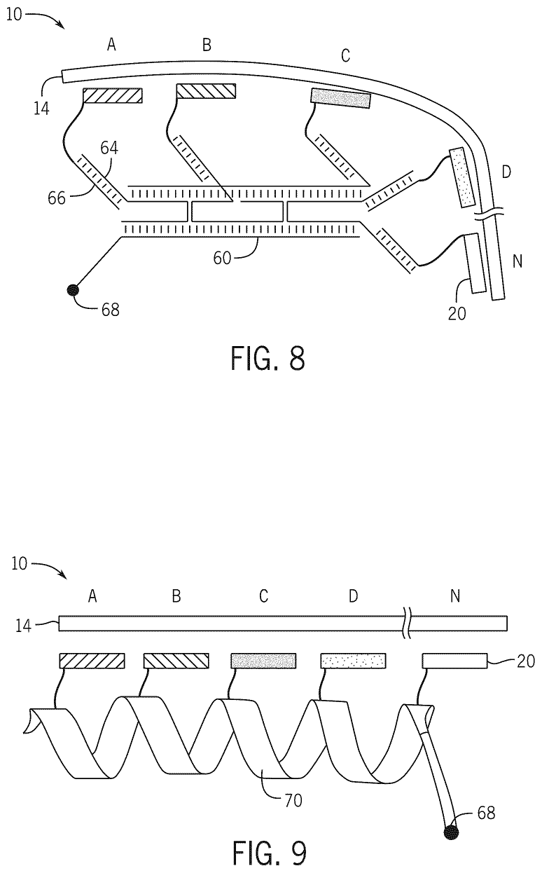

The surface 34 may be a bead 50 (see FIG. 5), such as a magnetic bead. In embodiments, the surface 34 may be a substrate, a flow cell surface, a planar surface, a shaped surface, a multiwell surface, a patterned surface, or a molecule such as a DNA tile structure (see FIG. 8) or a polypeptide (see FIG. 9).

The target binding for the set 12 includes multiple shorter binding interactions to correspondingly shorter respective target binding regions, which means that the multivalent assembly 10 has binding kinetics that more closely resemble the kinetics of shorter nucleotides. In an embodiment, the multivalent assembly 10 can achieve target specificity at or close to that or a conventional probe with improved binding kinetics characteristics of shorter nucleic acids.

As provided herein, an oligonucleotide set 12 or multivalent assembly 10 may include to two or more oligonucleotides 20, whereby each individual oligonucleotide 20 of the set 12 is complementary respective portion of the target nucleic acid 14, e.g., a single-stranded target nucleic acid 14. However, within a particular set 12, respective oligonucleotides 20 may have nonuniform or different lengths (e.g., different nucleotide lengths). The respective lengths of the oligonucleotides 20 can be selected to achieve a desired melting temperature (Tm) or to be within a particular melting temperature range.

FIG. 4 shows an example group of oligonucleotides having variable length but that nonetheless have melting temperatures that are within a particular Tm range such that a difference between a lowest Tm and a highest Tm of the set 12 is less than or equal to a predetermined value. In an embodiment, melting temperatures of the set 12 are within a twenty or ten degree Celsius range relative to one another. In an embodiment, the oligonucleotide lengths of the set 12 may be variable but also within a predetermined length range as well as having melting temperatures within a particular Tm range. In an embodiment, the longest oligonucleotide 20 of an individual set 12 may be 5-15 nucleotides or 5-10 nucleotides longer than a shortest oligonucleotide 20 of the individual set 12. In an embodiment, the oligonucleotides 20 of an individual set 12 may be between 20-30 nucleotides in length. In an embodiment, at least one oligonucleotide 20 of an individual set 12 has a different length relative to the other oligonucleotides 20 of the set 12. In an embodiment, every oligonucleotide 20 of an individual set 12 has a different length relative to the other oligonucleotides 20 of the set 12.

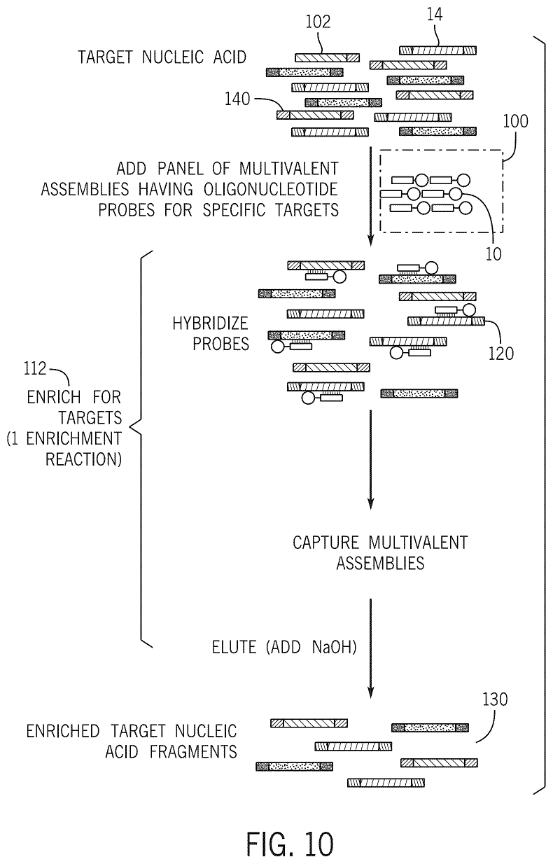

In an embodiment, the oligonucleotides 20 of an individual set 12 have similar Tms relative to one another of melting or separating from the target nucleic acid 14. Further, in embodiments in which a panel of multiple different probe sets 12 are used (see FIG. 10), all of the different sets 12 may be designed such that the all or most of the oligonucleotides 20 of each different probe set 12 fall within a preset estimated Tm range. In an embodiment, the estimated Tm range of the oligonucleotides is selected to be between 50-70° C. or between 55-65° C. In an embodiment, the estimated Tm range of the oligonucleotides is selected such that all nucleotides within the set 12 have melting temperatures within a 20 degree Celsius temperature range relative to one another. In an embodiment, the estimated Tm range of the oligonucleotides is selected such that all nucleotides within the set 12 have melting temperatures within a ten degree Celsius temperature range relative to one another. In an embodiment, the estimated Tm range of the oligonucleotides is selected such that all nucleotides within the set 12 are within 5-20 degrees Celsius of one another, 10-20 degrees Celsius of one another, or 15-20 degrees Celsius of one another. By making the probes different lengths or more variable lengths, a more uniform Tm between the probes can be achieved. Further, certain multivalent assemblies 10 may include modified nucleic acids (e.g., locked nucleic acids) to enhance stability and hybridization to the target nucleic acid 14.

In an embodiment, the Tm for a particular individual oligonucleotide 20 of the probe set 12, or a fully assembled multivalent assembly 10 may be estimated based on the following assumptions nearest neighbors formula:

$${T}_{m} = \frac{Δ H}{A + Δ S + R ln ( \frac{C}{4} )} - 273.15 + 16.6 log [ {Na}^{+} ]$$

### Where:

Tₘ=melting temperature in ° C.
ΔH=enthalpy change in kcal mol⁻¹ (accounts for the energy change during annealing/melting)
A=constant of −0.0108 kcal K⁻¹ mol⁻¹ (accounts for helix initiation during annealing/melting)
ΔS=entropy change in kcal K⁻¹ mol⁻¹ (accounts for energy unable to do work, i.e. disorder)
R=gas constant of 0.00199 kcal K⁻¹ mol⁻¹ (constant that scales energy to temperature)
C=oligonucleotide concentration in M or mol L⁻¹ (Table 1 is shown at 0.0000005, i.e. 0.5 μM)
−273.15=conversion factor to change the expected temperature in Kelvins to ° C.
[Na⁺]=sodium ion concentration in M or mol L⁻¹ (Table 1 is shown at 0.05, i.e. 50 mM)

TABLE 1
Enthalpy and entropy changes

| Molecule | |
|---|---|
| DNA | RNA |

| Interaction | ΔH | ΔS | ΔH | ΔS |
|---|---|---|---|---|
| AA/TT* | −9.1** | −0.0240 | −6.6 | −0.0184 |
| AT/TA | −8.6 | −0.0239 | −5.7 | −0.0155 |
| TA/AT | −6.0 | −0.0169 | −8.1 | −0.0226 |
| CA/GT | −5.8 | −0.0129 | −10.5 | −0.0278 |
| GT/CA | −6.5 | −0.0173 | −10.2 | −0.0262 |
| CT/GA | −7.8 | −0.0208 | −7.6 | −0.0192 |
| GA/CT | −5.6 | −0.0135 | −13.3 | −0.0355 |
| CG/GC | −11.9 | −0.0278 | −8.0 | −0.0194 |
| GC/CG | −11.1 | −0.0267 | −14.2 | −0.0349 |
| GG/CC | −11.0 | −0.0266 | −12.2 | −0.0297 |

Table 1:
*The left sequence is 5′ to 3′, while the right sequence is 3′ to 5′, e.g. for AA/TT, AA is 5′ to 3′ and TT is 3′ to 5′. When selecting values, always choose in the 5′ to 3′ direction, regardless of whether it is the left or right sequence in the correct orientation.
**Negative values reflect that annealing is enthalpically and entropically favorable. Positive values would reflect the reverse reaction, melting, and would lead to an identical Tₘ calculation.

In another example, the Tm may be estimated as follows:
For sequences less than 14 nucleotides the formula is:

$$Tm = ( wA + xT ) * 2 + ( yG + zC ) * 4$$

where w, x, y, z are the number of the bases A, T, G, C in the sequence, respectively.
For sequences longer than 13 nucleotides, the equation used is:

$$Tm = 64.9 + 41 * ( yG + zC - 16.4 ) / ( wA + xT + yG + zC )$$

Equations above assume that the annealing occurs under the standard conditions of 50 nM primer, 50 mM Na⁺, and pH 7.0. In an embodiment, the sequence length is based on a total length of the nucleic acid.

FIG. 5 shows an example multivalent assembly 10 in which the set 12 of oligonucleotides 20 are immobilized on a surface 34 of a bead 50. The illustrated example is a heteromultivalent arrangement in which multiple oligonucleotides having different sequences are immobilized onto a single particle (e.g., a magnetic bead). The number of unique probes per particle may be empirically but depends on variables including the size of the particle and the length of an optional linker 36, as shown in FIG. 6. The linker 36 provides flexibility and length away from the bead 50 so that the probes can arrange in the correct order to coordinate hybridization. The linker 36 may be a universal linker 36 that is a same linker even for different oligonucleotides. The linker 36 may be a nucleotide linker, a chemical linker, or a polymer linker. In an embodiment, the disclosed multivalent assemblies may include hybridization probe sets that, for example, may be used for target enrichment NGS. For hybridization capture, many particle types or multivalent assemblies (each with their own probe sets 12) are pooled together to enrich a desired group of target regions.

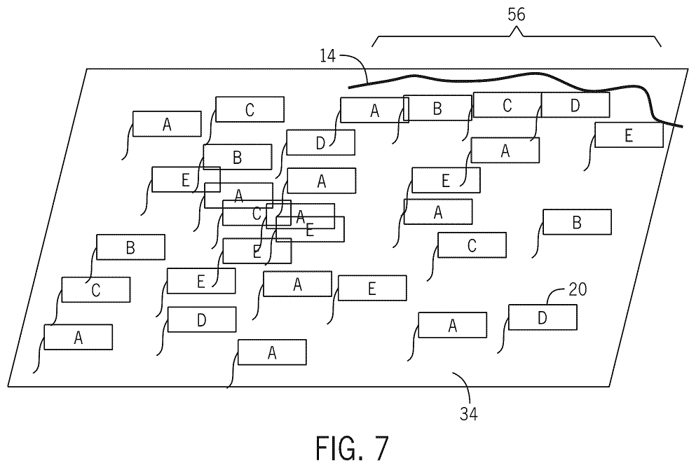

As illustrated in FIG. 7, the surface 34 of the bead or other structure may be randomly seeded with oligonucleotides 20 such that a subset 56 of the oligonucleotides 20 are arranged in a correct order relative to one another to facilitate hybridization to the target nucleic acid 14. The illustrated embodiment shows a single subset 56. However, depending on the surface density of the oligonucleotides 20, multiple subsets 56 may be formed on the surface 34. Thus, each bead 50 or other assembly 10 may be capable of hybridizing multiple targets, if available in a given sample. Each set 12 of oligonucleotides 20, in one embodiment, may include multiple copies of each individual oligonucleotide 20 representing different heterovalent binding sequences.

In FIG. 8, an example multivalent assembly 10 includes oligonucleotides 20 immobilized onto a pre-folded DNA tile 60. DNA origami techniques are used to assemble a tile base with variable hook arms 64 of different nucleotide sequences that are available for hybridizing to linker sequences 66 coupled to oligonucleotides 20. This allows for a single universal tile base 60 to be used for many different sets 12 in order to capture various different targets in a pooled manner. Additionally, hooks 64 can be arranged so that oligonucleotides 20 are arrayed sequentially to hybridize with targets according to their sequence. For hybridization capture, many tiles (each with their own probe sets 12) are pooled together to enrich a desired group of target regions. The tile 60 is coupled to an affinity molecule 68, such as a biotin, that binds to an affinity molecule binder, such as streptavidin, to facilitate separation of the bound target nucleic acids 14 from other components of a sample.

FIG. 9 shows an example multivalent assembly that includes a polypeptide scaffold 70. Oligonucleotides 20 can be attached to a universal peptide, e.g., the polypeptide scaffold 70, through multiple orthogonal chemistries in an arrayed manner. Peptide synthesis of an alpha helix with several orthogonal amino acid chemistry handles can be used to attach oligonucleotides 20 sequentially. Oligonucleotides 20 can be arrayed according to a target sequence in order to allow for target capture. Coupling to an affinity molecule 68 permits separation after hybridization. Several peptide:oligonucleotide multivalent assemblies 10, with specificity for respective different target nucleic acids 14 (e.g., having different sequences), can be pooled for parallel enrichment on desired target regions.

Multivalent assemblies 10 may include oligonucleotides 20 of a set 12 covalently attached at multiple branch points to a biotinylated scaffold, such as a dendrimer or bottlebrush. These multivalent assemblies 10 hybridize to their targets in solution and may be captured to streptavidin beads. In the bottlebrush approach, terminal deoxynucleotidyl transferase either extends azide-linked nucleotides from a ssDNA oligonucleotide or alternatively a DNA polymerase adds azide-linked nucleotides through amplifications of a template. After azide-linked nucleotide incorporation, the oligonucleotides 20 are covalently attached through azide-alkyne cycloaddition (click chemistry). Branched arrangements, which are coupled to more flexible scaffolds relative to beads, may lead to better coordination of probes to their respective target and also avoid settling or clumping.

FIG. 10 shows an example target enrichment workflow using the multivalent assemblies 10 as provide herein. The term enrichment or target enrichment refers to the process of increasing the relative abundance of particular nucleic acid sequences in a sample relative to the level of nucleic acid sequences as a whole initially present in said sample before treatment. Thus, the enrichment step provides a percentage or fractional increase rather than directly increasing for example, the copy number of the nucleic acid sequences of interest as amplification methods, such as PCR, would. The methods as described herein may be used to remove DNA strands that are not desired to be sequenced, rather than to specifically amplify only the sequences of interest. At the level of the whole genome, removing 50% of the DNA sample gives a two-fold reduction in the cost and time of sequencing the remaining regions of biological interest from the whole genome. The methods as described herein can also be used to select large regions of a genome (e.g., megabases) for resequencing of multiple individuals, or can select out all the exons in a genomic sample. The synthesis of one array, or pool of oligonucleotides, can be used to process multiple samples of interest, and thus the costs of the oligonucleotide synthesis can be amortized over many individual samples.

The illustrated target enrichment workflow uses a panel 100 of multivalent assemblies 10 specific for respective different target nucleic acids 14. The panel 100 may include multivalent assemblies 10 that are capable of hybridizing to a selected group of different target sequences. The target sequences targeted by the panel may include whole-exome sequencing, or predesigned or custom sequencing panels for diagnostics or screening, environmental monitoring, infectious disease surveillance, etc. Thus, each multivalent assembly includes a set 12 of unique oligonucleotides 20 with sequences specific for a particular target sequence 14. Thus, the hybridization sequences of individual oligonucleotides 20 of the set 12 may all be unique within the panel 100. In the illustrated example, the oligonucleotides 20 may function as hybridization probes.

As illustrated, the target nucleic acids 14 may be in the form of nucleic acid fragments 102. Nucleic acid fragments 102 as provided herein, such as for target enrichment or amplification reactions, may include sequence fragments that are relatively large, such as 10 kilobases (kb)-62 megabases (Mb) in length. In other embodiments, the fragments that are less than about 1 kb in length, e.g., in the range 100-1000 bases in length or in the range of from 450-750 bases in length. It would be apparent to the skilled artisan that the following non-limiting fragmentation methods may be used: restriction endonucleases, other suitable enzymes, tagmentation via transposases, mechanical forms of fragmentation, such as nebulisation or sonication, or non-enzymatic chemical fragmentation.

The panel 100 and fragments 102 are contacted with one another at a hybridization step 112 under conditions to permit hybridization of the oligonucleotides 20 of the multivalent assemblies 10 to their respective target nucleic acids 14. Hybridization results in forming multivalent assembly-target nucleic acid complexes 120 for at least some of the fragments 102 and at least some of the multivalent assemblies 10. That is, the hybridization occurs when a target nucleic acid 14 is present within the fragments 102. Further, some of the fragments 102 may not have any sequences that are targets of the panel 100. In an embodiment, the hybridization (e.g., binding and/or assembly of the set 12) to the target nucleic acid 14 as provided herein occurs at 50° C.-65° C. and with hybridization times of three hours or less, two hours or less, or an hour or less to achieve desired levels of target nucleic acid binding and avoid nonspecific binding.

The hybridization step 112 may include a denaturation step in which the fragments 102 and the probe panel 100 are heated to at least 90° C. (e.g., 90° C.-95° C.) to denature the fragments 102 and to separate the nucleic acids of the multivalent assemblies 10. The workflow may include a gradual or stepwise temperature decrease into the desired hybridization temperature range. In one example, after denaturation, the temperature is lowered to be at or below the melting temperature of the individual oligonucleotides (e.g. 50° C.-65° C.). This relatively lower temperature permits binding of the individual oligonucleotides 20 of the set 12. The 50° C.-65° C. is held for a period of time (e.g., 10-20 minutes) that is relatively short. The temperature is slowly increased so that any non-specifically bound probes melt off. Again, this relatively higher temperature is held for a predetermined period of time that can be relatively short (e.g., 10-20 minutes).

The hybridization step 112 may be performed either on the solid surface 34, such as on beads 50, or in solution. In certain embodiments, at least one nucleic acid of the multivalent assemblies 10 may have modifications or an affinity binder 68 that facilitate separation of bound fragments 102 from the unbound fragments 102. Accordingly, the multivalent assemblies 10 as provided herein may be coupled to an affinity binding molecule 68 of a binding pair, for example biotin/streptavidin, biotin/avidin, biotin/neutravidin, DNP/anti-DNP, DIG/anti-DIG, etc. and a specific antibody that binds digoxigenin are examples of specific binding pairs. In one example, biotinylation of the nucleic acid of the multivalent assemblies 10 facilitates selection via streptavidin (e.g., streptavidin beads). The affinity binding molecule 68 may be an antibody ligand capable of being conjugated to a nucleotide. In certain embodiments, the modification is provided at the 5′ or the 3′ end of an individual nucleic acid of the multivalent assemblies 10. Nucleic acids of the multivalent assemblies 10 may also include unique barcodes or sequences (e.g., unique molecular identifiers) that facilitate identification. In embodiments in which the multivalent assemblies 10 include a bead 50, the bead 50 may be a magnetic bead that is capable of being separated using magnetic pulldown. In other embodiments, the bead 50 may include an affinity binder 68.

In certain embodiments, the hybridization step 112 may be performed in solution, and subsequent addition of beads having the mating affinity binder results in binding of affinity-binder-carrying multivalent assemblies 10, either as duplexes with the fragments, or as single strands. For example, when the multivalent assemblies 10 include DNA or polypeptide scaffolds coupled to affinity binders. The multivalent assemblies 10 can hybridize to fragments 102 including the target sequence in solution, and the multivalent assemblies 10 can be captured via the affinity binder. Uncaptured fragments 102 can be removed from the beads by washing, for example.

In one embodiment the captured fragments can be removed from the probe-target complex prior to sequencing for example by elution. Removal by denaturation of the selected targets from the immobilized capture probes will generally give a solution of enriched target nucleic acid fragments 130. The enriched target nucleic acid fragments 130 can be provided for subsequent sequencing steps. In an alternative embodiment the enriched target nucleic acid fragments 130 may be amplified while still attached to the beads by, for example, emulsion phase PCR, or may be eluted from the beads and amplified in solution prior to surface attachment as part of a sequencing reaction.

In one embodiment, the fragments 102 may be fragments generated through a library preparation workflow and that include end adaptors 140 suitable for use in sequencing, e.g., that can capture the enriched target nucleic acid fragments 130 on a solid support and that can serve as primer binding sites. The adaptors 140 may be universal adaptors, e.g., that include common sequences. In an embodiment, the adaptors 140 may be Illumina sequencing adaptors (Illumina, Inc.). Accordingly, the common sequences of the adaptors 140 may tend to hybridize to one another. In such an embodiment, adaptor blockers may also be used during the hybridization step 112. The fragments may be from a single library (i.e., singleplex) or may be multiplexed from multiple libraries.

In another embodiment, the adaptors 140 may be ligated to the enriched target nucleic acid fragments 130 after elution and additional preparation steps. For example, the enriched target nucleic acid fragments 130 may also be further fragmented after elution from the beads 120 or other support. In an embodiment, it may be advantageous to capture relatively larger fragments 102, e.g., having an average size of 10 kB, and thereby require fewer probe sets 12 to select out a specific megabase region. A 10 kB region can be selected, but not easily amplified, and therefore further fragmentation, to an average of a few hundred bases may be used after the enrichment step. If a second fragmentation step is used, then the universal adaptors 140 can be ligated onto the enriched target nucleic acid fragments 130 after the elution and after the further fragmentation step.

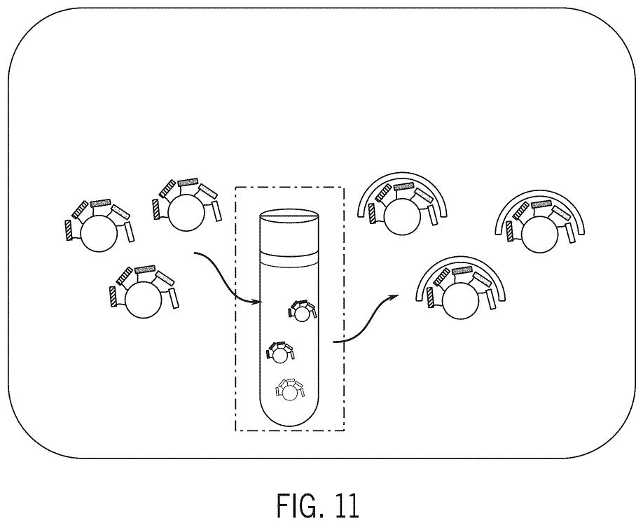

In another embodiment, the disclosed multivalent assemblies 10 may be used for nucleic acid capture. FIG. 11 shows an example of capture of cell-free DNA and/or RNA from biological samples via bead-based multivalent assemblies 10. Capturing nucleic acids from biological samples using multivalent beads or other multivalent assemblies 10 offers advantages to traditional purification approaches such as column-based purification. The multivalent assemblies 10 couple purification and enrichment of target fragments. In another example, the probes can be used to deplete unwanted nucleic acids such as abundant RNAs like ribosomal RNAs from samples. The multivalent assemblies 10, due to their high avidity, can capture target fragments with higher sensitivity at lower copy numbers compared to traditional approaches. Once the nucleic acids are captured onto multivalent assemblies 10, the captured nucleic acids could be enzymatically manipulated to add sequences such as the sequencing adapters on the bead surface. This would enable enzyme and buffer exchanges while the nucleic acids are coupled to beads.

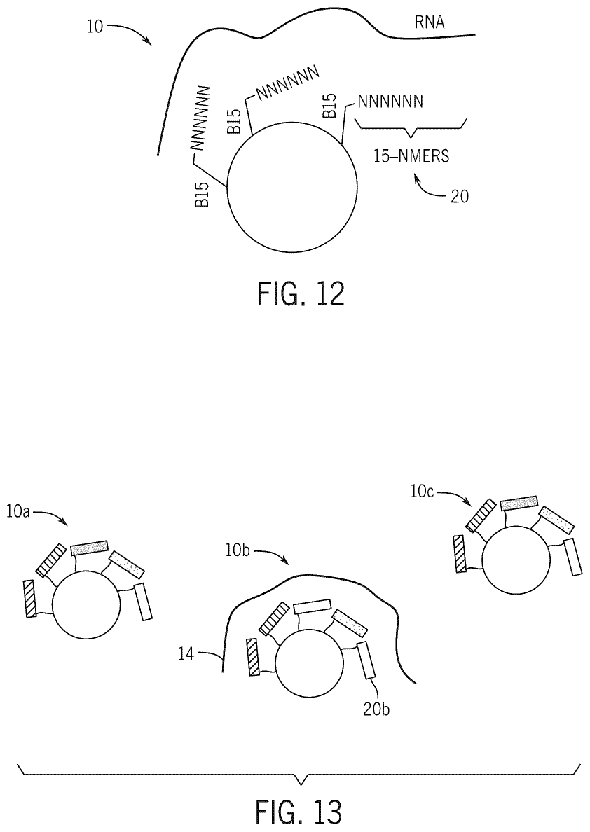

The multivalent assemblies 10 may be used in converting RNA to cDNA (i.e., cDNA conversion) on beads. FIGS. 12-13 depict nucleic acid capture via multivalent beads to convert RNA into cDNA libraries. Traditional cDNA synthesis use hexamers to convert bulk RNA to cDNA. Hexamers have low melting temperatures that lead to lower synthesis of cDNA from template RNA (40%). Multivalent beads may be used to improve cDNA conversion. FIG. 12 shows examples of oligonucleotide N-mers (randomers) for bulk cDNA synthesis, while FIG. 13 shows specific oligonucleotide probes in targeted cDNA synthesis. In FIG. 13, the target nucleic acid 14, here an RNA, binds the multivalent assembly 10b having oligonucleotide probes 20b specific for (complementary to) the target nucleic acid 14 while the other multivalent assemblies 10a, 10c that do not have probes specific for the target nucleic acid 14 remain unbound. The bound target 14 can be used for cDNA synthesis from the probes, e.g., the probes 20b. Covalently attached cDNA also offers additional benefits including physical separation enabling compartmentalization for RNA/DNA co-assay, enrichment or depletion of target RNAs, and buffer/enzymatic exchanges (single tube assay)

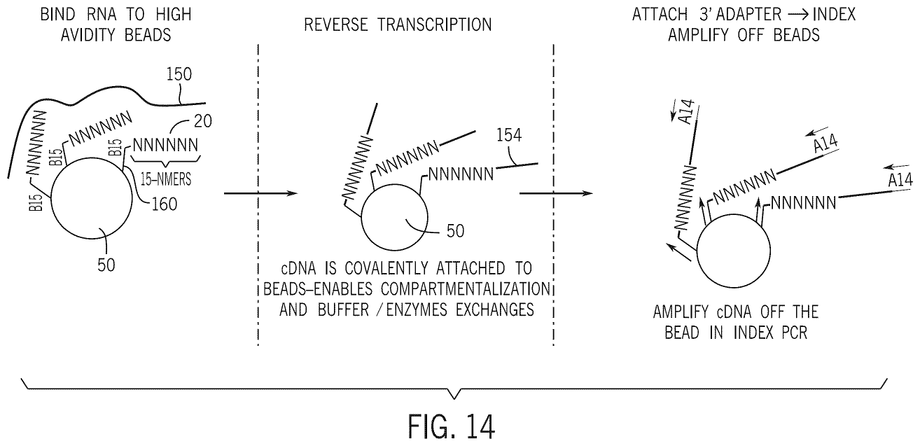

FIG. 14 shows a reverse transcription workflow for cDNA synthesis using multivalent assemblies 10 as shown in FIG. 12. After capture of an RNA molecule 150 (e.g., a single-stranded RNA or a messenger RNA) using oligonucleotides 20, the complementary strand 154 is extended from 3′ ends of the immobilized oligonucleotides 20, e.g., using reverse transcriptase. The oligonucleotides 20 are shown as 15 mers, but other lengths are also contemplated, such as between 6-30 bases, 10-20 bases, 15-25 bases in length. The resultant extended complementary cDNA strand is, therefore, coupled to the bead 5. In an embodiment, the oligonucleotides 20 may include a universal adapter sequence 160 at a 5′ end as part of a linker sequence. Following extension of the complementary strands 154, the strand 3′ ends can be adapterized via addition of a 3′ adapter (e.g., via ligation or amplification with a primer having the complement of the desired 3′ adapter). The immobilized strands can be amplified as part of a library preparation step, and the amplified strands can be provided as input to an NGS sequencing reaction.

### EXAMPLES

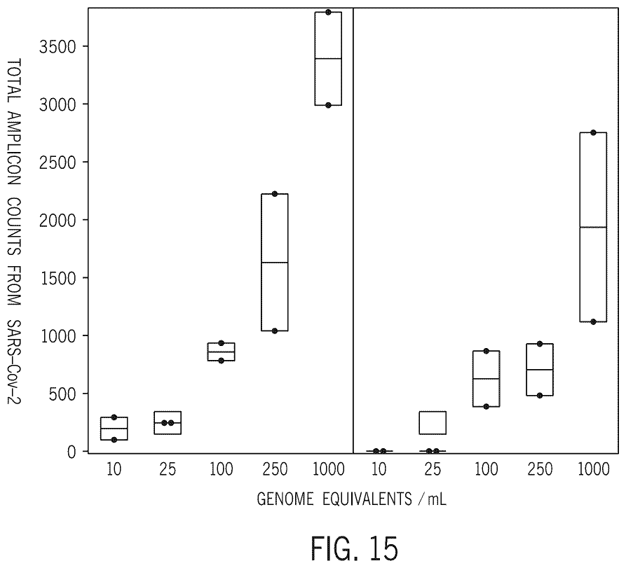

FIG. 15 shows the performance of hybridization-based extraction surpasses total nucleic acid extraction. Multivalent bead assemblies were compared to standard purification method using a commercially available kit in the COVID-Seq assay. The multivalent beads improved sensitivity and reduced the number of steps and touchpoints required. This was conducted without optimization of beads or oligonucleotide probe design, but still demonstrated a clear benefit for capturing low copy number nucleic acids from non-purified sample.

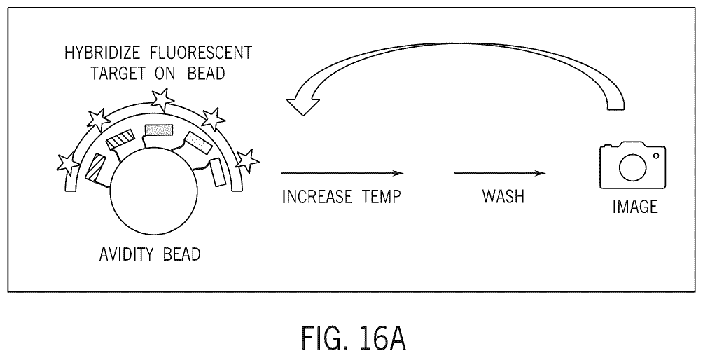

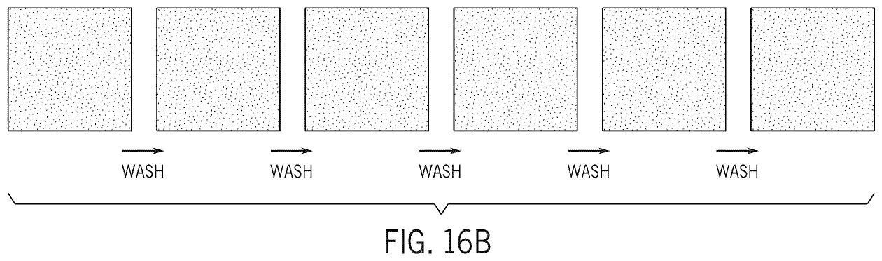

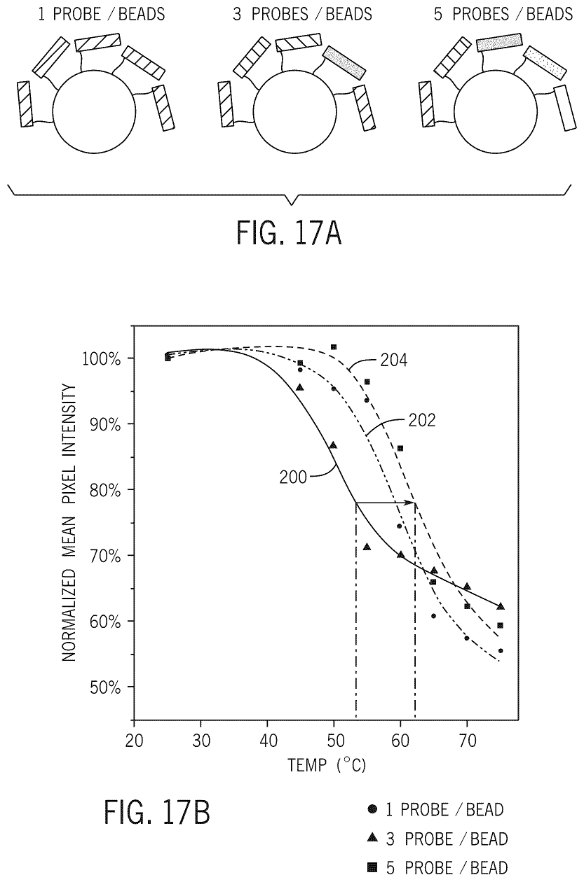

FIGS. 16-17 show an experimental workflow and results to measure the use of melting temperature as a proxy for increased avidity. A 400 base pair internally labeled (Cy5) single-stranded amplicon was used as a target, and oligonucleotide probes of 20 bp each plus a linker (20 Ts) were designed to capture the target. The designed probes were coupled to NeutrAvidin magnetic beads. Measuring the melting temperature of oligonucleotide/target duplexes was used to compare the binding strength or avidity of multivalent beads. FIG. 17A shows example bead constructs. In FIG. 17B, the melting temperatures of beads having 1 probe type per bead (“1 probe type bead”), 3 probe types per bead (“3 probe type bead”), and 5 probe types per beads (“5 probe type bead”) were quantified. The probe type refers to a unique oligonucleotide sequence. Accordingly, a 5 probe type bead includes 5 different oligonucleotide probes having respective different sequences while a 1 probe type bead includes a single probe type. However, each bead may include multiple copies of each different probe type, where applicable, or the single probe type. If the probes were not binding in coordination, the melting temperature of single probe type beads should be the same as the melting temperature of 5 probe type beads. If coordination is present, then the melting temperature of 1 probe type beads would be less than 5 probe type beads. This coordination was observed, with the single probe type having a lower melting temperature relative to the 3 probe type beads and 5 probe type beads, showing enhanced avidity with beads containing 3 and/or 5 oligonucleotide types.

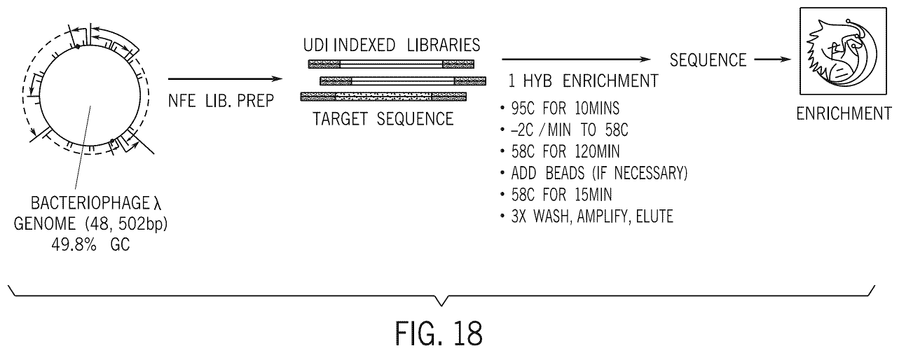

FIG. 18 shows an enrichment protocol suitable for bead enrichment. Lambda phage libraries were generated using a NexteraFlex library prep protocol in the NexteraFlex for enrichment kit followed by a modified enrichment protocol in the NexteraFlex for enrichment kit. The modification was added avidity beads coupled to probes (as provided herein) instead of in-solution probes. The avidity beads were added after the initial 95° C. and ramp down to 56° C. The last modification is skipping the conventional capture step after enrichment.

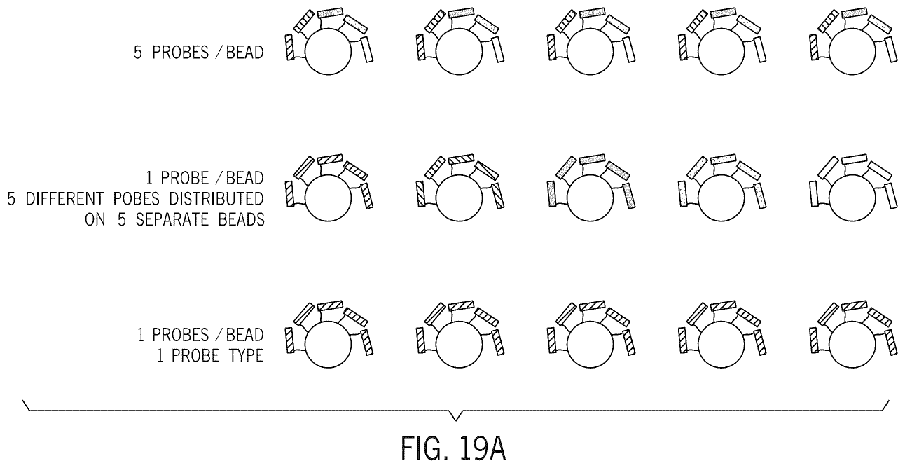

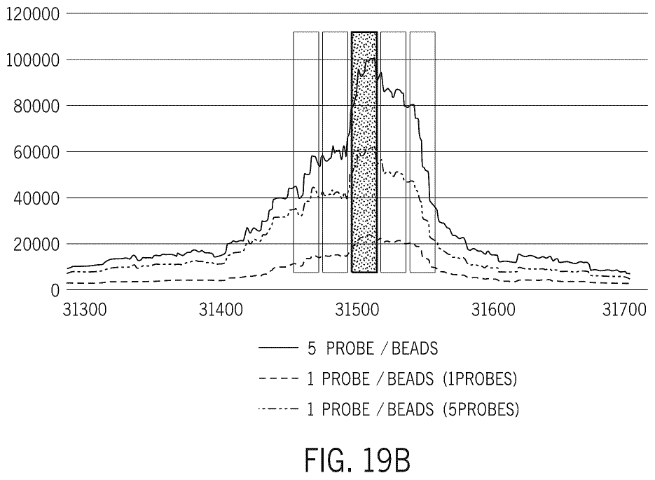

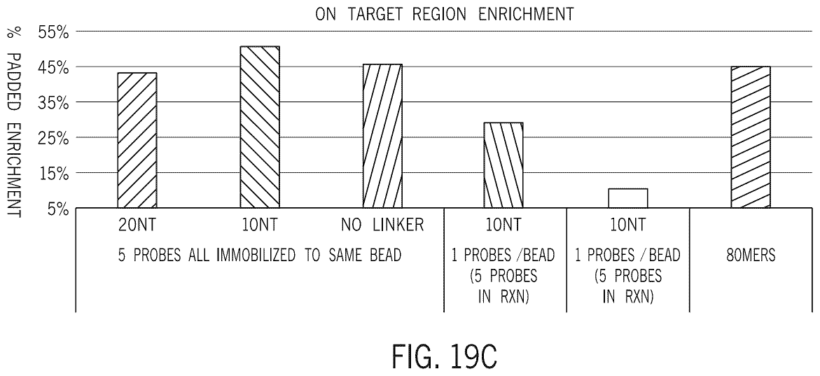

FIG. 19A shows schematic illustration of heteromultivalent assemblies with 5 different probes (e.g., 5 different probe sequences) attached to a single beads vs 5 different probe sequences separated on respective different beads vs 1 probe sequence on one bead was conducted. A set of 5 probes approximately 20 nucleotides in length with a 5′ biotin poly A linker were designed. It should be understood that the 5 different probe types refer to available sequences, and each bead may have multiple copies of the available sequence. Accordingly, a bead with 5 different probe types may have multiple copies of each of the 5 different oligonucleotide sequences of the probes on the bead while a bead with a single probe type may have multiple copies of the single probe type. The multivalent beads were compared to in solution 80 mers aligned to the same region. Using the workflow mentioned in FIG. 18, higher on target enrichment was observed with all 5 probes on a single bead compared to each probe separated on different beads, as shown in FIGS. 19B and 19C. FIG. 19C graph shows the percent of library fragments that overlap the target region within 500 bp. The left three columns show high level of enrichment with 5 probes immobilized onto the same beads and this high level is not dependent on linker length, and three different linker lengths (20 nt, 10 nt, 0 nt) were compared. The red column is the enrichment from 5 probes on 5 different beads, which has less efficient enrichment. And the green bar shows the worse enrichment with only 1 of the 5 probes in the enrichment. The grey bar is the comparison to an 80 nt probe. This provided evidence that enhanced avidity improves on target capture efficiency.

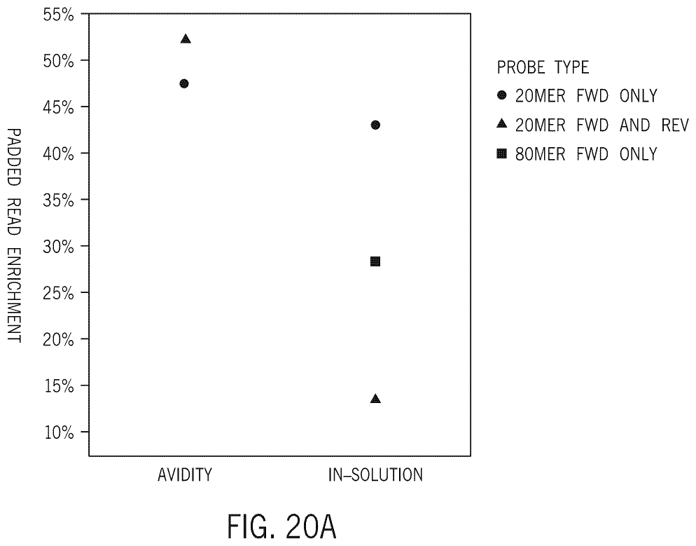

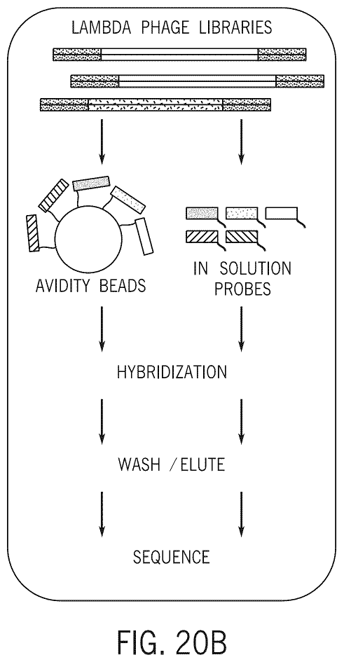

FIG. 20A shows that multivalent beads improve read enrichment and enable hybridization to both strands. Forward (FWD) or reverse (REV, reverse compliment of FWD) on separate beads or in solution were evaluated relative to a contiguous or nonmultivalent case of an 80 mer FWD only oligonucleotide probe using a lambda phage model. An enhancement relative to the 80 mer oligonucleotide probe for on target enrichment was observed on beads and a suppression in solution. FIG. 20B is a schematic illustration of the experimental workflow.

The terms target or target nucleic acid as disclosed herein may refer to nucleic acids having sequences of interest that hybridize to the oligonucleotides of a multivalent assembly as provided herein. The term includes nucleic acid sequences for which a sub-sequence of which binds to the multivalent assembly. In an embodiment, the target nucleic acid is a fragment generated as part of an NGS workflow.

The oligonucleotides disclosed herein hybridize to target nucleic acids in a nucleic acid sample. The hybridization is between single stranded nucleic acid sequences. This can be achieved by a number of well-known methods in the art such as, for example using heat to denature or separate complementary strands of double stranded nucleic acids, which on cooling can hybridize to the probes. Further, when the sets 12 are provided as part of a probe panel (e.g., panel 100) in which each different target region is spatially separate, the probe panel may be stored together and provide in a single reaction vessel. Alternatively, individual probe sets of the probe panel may be stored separately.

The oligonucleotides, e.g., probes, as disclosed herein are nucleic acids capable of binding to a target nucleic acid through one or more types of chemical bonds, usually through complementary base pairing, usually through hydrogen bond formation. The oligonucleotides may include natural or modified bases and may be RNA or DNA. In addition the bases may be joined by a linkage other than a phosphodiester bond so long as it does not interfere with hybridization. Thus oligonucleotides may also be peptide nucleic acids (PNA) in which the constituent bases are joined by peptide bonds rather than phosphodiester linkages.

The target nucleic acid can be derived from any in vivo or in vitro source, including from one or multiple cells, tissues, organs, or organisms, whether living or dead, or from any biological or environmental source (e.g., water, air, soil). For example, in some embodiments, the target nucleic acid comprises or consists of eukaryotic and/or prokaryotic dsDNA that originates or that is derived from humans, animals, plants, fungi, (e.g., molds or yeasts), bacteria, viruses, viroids, mycoplasma, or other microorganisms. In some embodiments, the target nucleic acid comprises or consists of genomic DNA, subgenomic DNA, chromosomal DNA (e.g., from an isolated chromosome or a portion of a chromosome, e.g., from one or more genes or loci from a chromosome), mitochondrial DNA, chloroplast DNA, plasmid or other episomal-derived DNA (or recombinant DNA contained therein), or double-stranded cDNA made by reverse transcription of RNA using an RNA-dependent DNA polymerase or reverse transcriptase to generate first-strand cDNA and then extending a primer annealed to the first-strand cDNA to generate dsDNA. In some embodiments, the target nucleic acid comprises multiple dsDNA molecules in or prepared from nucleic acid molecules (e.g., multiple dsDNA molecules in or prepared from genomic DNA or cDNA prepared from RNA in or from a biological (e.g., cell, tissue, organ, organism) or environmental (e.g., water, air, soil, saliva, sputum, urine, feces) source. In some embodiments, the target nucleic acid is from an in vitro source. For example, in some embodiments, the target nucleic acid comprises or consists of dsDNA that is prepared in vitro from single-stranded DNA (ssDNA) or from single-stranded or double-stranded RNA (e.g., using methods that are well-known in the art, such as primer extension using a suitable DNA-dependent and/or RNA-dependent DNA polymerase (reverse transcriptase). In some embodiments, the target nucleic acid comprises or consists of dsDNA that is prepared from all or a portion of one or more double-stranded or single-stranded DNA or RNA molecules using any methods known in the art, including methods for: DNA or RNA amplification (e.g., PCR or reverse-transcriptase-PCR (RT-PCR), transcription-mediated amplification methods, with amplification of all or a portion of one or more nucleic acid molecules); molecular cloning of all or a portion of one or more nucleic acid molecules in a plasmid, fosmid, BAC or other vector that subsequently is replicated in a suitable host cell; or capture of one or more nucleic acid molecules by hybridization, such as by hybridization to DNA probes on an array or microarray. Target nucleic acids as provided herein may include, but are not limited to DNA, RNA, peptide nucleic acid, morpholino nucleic acid, locked nucleic acid, glycol nucleic acid, threose nucleic acid, mixtures thereof, and hybrids thereof. In an embodiment, genomic DNA fragments, or amplified copies thereof, are used as the target nucleic acid. In another embodiment, mitochondrial or chloroplast DNA is used. Still other embodiments are targeted to RNA or derivatives thereof such as mRNA or cDNA. In some embodiments, target nucleic acid can be from a single cell. In some embodiments, target nucleic acid can be from acellular body fluids, for example, plasma or sputum devoid of cells. In some embodiments, target nucleic acid can be from circulating tumor cells.

The disclosed multivalent assemblies may include synthetic sequences or non-naturally occurring sequences that are not specific for any target sequences of the target source to reduce non-specific binding.

This written description uses examples, including the best mode, and also to enable any person skilled in the art to practice the disclosed embodiments, including making and using any devices or systems and performing any incorporated methods. Such other examples are intended to be within the scope of the claims if they have structural elements that do not differ from the literal language of the claims, or if they include equivalent structural elements with insubstantial differences from the literal languages of the claims.

What is claimed is:

1. A multivalent assembly, comprising:
a first single-stranded oligonucleotide probe complementary to a first region of a target nucleic acid;
a second single-stranded oligonucleotide probe complementary to a second region of the target nucleic acid; and
a third single-stranded oligonucleotide probe complementary to a third region of the target nucleic acid, and wherein melting temperatures of the first single-stranded oligonucleotide probe, the second single-stranded oligonucleotide probe, and the third single-stranded oligonucleotide probe from the target nucleic acid are all within a 20 degrees Celsius range.

2. The multivalent assembly of claim 1, wherein the first single-stranded oligonucleotide probe, the second single-stranded oligonucleotide probe, and the third single-stranded oligonucleotide probe are 20-30 nucleotides in length.

3. The multivalent assembly of claim 1, wherein the first single-stranded oligonucleotide probe, the second single-stranded oligonucleotide probe, and the third single-stranded oligonucleotide probe are immobilized on a surface via respective linkers.

4. The multivalent assembly of claim 3, wherein the respective linkers comprise a universal adapter sequence.

5. The multivalent assembly of claim 3, wherein the surface comprises a bead surface or a planar surface.

6. The multivalent assembly of claim 5, wherein the bead surface comprises multiple copies of the first single-stranded oligonucleotide probe, the second single-stranded oligonucleotide probe, and the third single-stranded oligonucleotide probe.

7. The multivalent assembly of claim 1, wherein the first single-stranded oligonucleotide probe, the second single-stranded oligonucleotide probe, and the third single-stranded oligonucleotide probe are coupled to a polypeptide.

8. The multivalent assembly of claim 1, wherein the first single-stranded oligonucleotide probe, the second single-stranded oligonucleotide probe, and the third single-stranded oligonucleotide probe are coupled to a nucleic acid tile.

9. The multivalent assembly of claim 1, wherein the first region, the second region, and the third region of the target nucleic acid are contiguous.

10. The multivalent assembly of claim 1, wherein the first region and the second region of the target nucleic acid are separated by 5 or fewer nucleotides.

11. The multivalent assembly of claim 10, wherein the second region and the third region of the target nucleic acid are separated by 5 or fewer nucleotides.

12. The multivalent assembly of claim 1, comprising a fourth single-stranded oligonucleotide probe comprising a fourth probe capture region complementary to a fourth region of the target nucleic acid, wherein a melting temperature of the fourth single-stranded oligonucleotide probe from the target nucleic acid is within the 20 degrees Celsius range.

13. The multivalent assembly of claim 12, comprising a fifth single-stranded oligonucleotide probe comprising a fifth probe capture region complementary to a fifth region of the target nucleic acid, wherein a melting temperature of the fifth single-stranded oligonucleotide probe from the target nucleic acid is within the 20 degrees Celsius range.

14. The multivalent assembly of claim 1, wherein the first single-stranded oligonucleotide probe, the second single-stranded oligonucleotide probe, and the third single-stranded oligonucleotide probe are different lengths relative to one another.

15. A multivalent bead assembly, comprising:
a bead surface;
first single-stranded oligonucleotide probes comprising a first hybridization sequence complementary to a first region of a target nucleic acid;
second single-stranded oligonucleotide probes comprising a second hybridization sequence complementary to a second region of the target nucleic acid; and
third single-stranded oligonucleotide probes comprising a third hybridization sequence complementary to a third region of the target nucleic acid, wherein the first single-stranded oligonucleotide probes, the second single-stranded oligonucleotide probes, and the third single-stranded oligonucleotide probes are immobilized randomly on the bead surface, and wherein the first hybridization sequence, the second hybridization sequence, and the third hybridization sequence are different from one another.

16. The multivalent bead assembly of claim 15, wherein the first single-stranded oligonucleotide probes, the second single-stranded oligonucleotide probes, and the third single-stranded oligonucleotide probes are 20-30 nucleotides in length.

17. The multivalent bead assembly of claim 15, wherein the first single-stranded oligonucleotide probes, the second single-stranded oligonucleotide probes, and the third single-stranded oligonucleotide probes are immobilized on the bead surface via respective linkers.

18. The multivalent bead assembly of claim 15, wherein the respective linkers comprise universal linkers.

19. The multivalent bead assembly of claim 15, wherein the first region, the second region, and the third region of the target nucleic acid are contiguous.

20. The multivalent bead assembly of claim 15, wherein the first region and the second region of the target nucleic acid are separated by 5 or fewer nucleotides.

21. The multivalent bead assembly of claim 15, wherein the second region and the third region of the target nucleic acid are separated by 5 or fewer nucleotides.

22. The multivalent bead assembly of claim 15, wherein oligonucleotide probes to different target nucleic acids are not immobilized on the bead surface.

23. The multivalent bead assembly of claim 15, wherein a subset of the first single-stranded oligonucleotide probes, the second single-stranded oligonucleotide probes, and the third single-stranded oligonucleotide probes are arranged on the bead surface such that the target nucleic acid hybridizes to an individual first single-stranded oligonucleotide probe, an individual second single-stranded oligonucleotide probe, and an individual third single-stranded oligonucleotide probe.

24. The multivalent bead assembly of claim 15, comprising the target nucleic acid.

25. The multivalent bead assembly of claim 24, wherein the target nucleic acid is an RNA molecule.

26. The multivalent bead assembly of claim 15, wherein the first single-stranded oligonucleotide probes, the second single-stranded oligonucleotide probes, and the third single-stranded oligonucleotide probes are randomers.

27. The multivalent bead assembly of claim 15, wherein the first single-stranded oligonucleotide probes are a different length than one or both of the second single-stranded oligonucleotide probes and the third single-stranded oligonucleotide probes.

28. A hybridization kit, comprising:
a first bead comprising:
a first plurality of single-stranded oligonucleotide probes randomly immobilized on a surface of the first bead, wherein a first subset of the first plurality are complementary to a first region of a first target nucleic acid, a second subset of the first plurality are complementary to a second region of the first target nucleic acid, and a third subset are complementary to a third region of the first target nucleic acid; and
a second bead comprising:
a second plurality of single-stranded oligonucleotide probes randomly immobilized on a surface of the second bead, wherein a first subset of the second plurality are complementary to a first region of a second target nucleic acid, a second subset of the second plurality are complementary to a second region of the second target nucleic acid, and a third subset of the second plurality are complementary to a third region of the second target nucleic acid.

29. The hybridization kit of claim 28, wherein the first bead and the second bead are part of a pool of beads.

30. The hybridization kit of claim 28, wherein a fourth subset of the first plurality are complementary to a fourth region of the target nucleic acid, and wherein a fifth subset of the first plurality are complementary to a fifth region of the target nucleic acid.

31. The hybridization kit of claim 28, wherein the first target nucleic acid and the second target nucleic acid are complementary to one another.

32. A method of target enrichment, comprising:
contacting nucleic acid fragments with a plurality of multivalent assemblies to form multivalent assembly-target nucleic acid complexes, wherein the multivalent assemblies comprise individual probe sets specific for respective target nucleic acids, an individual probe set comprising:
a first oligonucleotide probe complementary to a first subregion of a target nucleic acid;
a second oligonucleotide probe complementary to a second subregion of the target nucleic acid; and
a third oligonucleotide probe complementary to a third subregion of the target nucleic acid, wherein the first oligonucleotide probe, the second oligonucleotide probe, and the third oligonucleotide probe have sequences that are distinguishable from one another; and
separating the multivalent assembly-target nucleic acid complexes from unhybridized nucleic acid fragments of the nucleic acid fragments to generate separated nucleic acid fragments.

33. The method of claim 32, comprising sequencing the purified nucleic acid fragments.

34. The method of claim 32, comprising adding adaptors to ends of the nucleic acid fragments before the contacting.

35. The method of claim 32, wherein an individual multivalent assembly comprises the individual probe set immobilized on a surface of a bead.

36. The method of claim 32, wherein the separating comprises a magnetic separation to capture the bead.

37. The method of claim 32, wherein the separating comprises a capture of an affinity binder of the multivalent assembly.

38. The method of claim 32, wherein an individual multivalent assembly comprises the individual probe set immobilized on a DNA tile.

39. The method of claim 32, wherein an individual multivalent assembly comprises the individual probe set immobilized on a polypeptide scaffold.

40. The method of claim 32, comprising generating the nucleic acid fragments.

41. A method of cDNA synthesis, comprising:
contacting an RNA sample with a multivalent assembly to capture an RNA molecule, the multivalent assembly comprising a probe set immobilized on a surface of a bead, wherein the probe set comprises:
a first oligonucleotide probe complementary to a first subregion of the RNA molecule;
a second oligonucleotide probe complementary to a second subregion of the RNA molecule; and
a third oligonucleotide probe complementary to a third subregion of the RNA molecule, wherein the first oligonucleotide probe, the second oligonucleotide probe, and the third oligonucleotide probe have sequences that are distinguishable from one another; and
extending the first oligonucleotide probe using a reverse transcriptase to generate a cDNA complementary to at least a portion of the RNA molecule.

42. The method of claim 41, wherein the first oligonucleotide probe, the second oligonucleotide probe, and the third oligonucleotide probe are random Nmers at least 15 bases in length.
